(19) United States
(12) Patent Application Publication
(10) Pub. No.: US 2005/0137462 A1
(43) Pub. Date: Jun. 23, 2005

(54) Posture sensing apparatus

(76) Inventor: Myoung-Ho Cho, Seoul (KR)

Correspondence Address: BURNS DOANE SWECKER & MATHIS L L P, POST OFFICE BOX 1404, ALEXANDRIA, VA, 22313-1404, US

(21) Appl. No.: 10/505,859

(22) PCT Filed: Feb. 27, 2002
(86) PCT No.: PCT/KR02/00335

(51) Int. Cl. A61B 5/00

(52) U.S. Cl. 600/300

(57) ABSTRACT

Posture sensing apparatus for notifying an occupant of the posture of the occupant comprises a sensing device and sitting means for infants, babies and children: The sensing device comprises sensors, microcomputer, responding means, switch part and power supply that are electrically interconnected to one another. The sensors generate/send signals to the microcomputer when an occupant on the sitting means contacts thereto. The microcomputer orders the responding means either to produce responding signal or not, according to predetermined program, and when the responding means responds, it is arranged such that the response is stopped when the occupant corrects the posture and contacts all the sensor(s).

### TECHNICAL FIELD

The present invention relates to a sitting means, and particularly relates to a sitting means having sensing device for sensing the posture of an occupant, and thereby for notifying an occupant and/or the attendant to the occupant of the posture of the occupant, and encouraging the occupant and/or the attendant to correct the posture of the occupant.

### BACKGROUND ART

Generally, a “sitting means” such as chairs, seats and the like has a seat member that supports the thighs and pelvis of an occupant and a back member that is adapted to provide support to the back of the occupant.

A “baby holding means” such as child car seats also has a bottom seat portion and a backrest portion that support the thighs, pelvis and the back of an occupant.

The baby holding means may be any one of a number of such known devices as an infant seat, child car seat, child car bed, stroller, infant bed, hammock, cradle, rocker, bassinet, shopping cart, infant carrier, baby carrying pack, swing, crib, walker, baby jogger, high chair, stationary infant stand and the like. Each of the baby holding means is provided as a separate unit and some are designed to function as a combination of a few of these devices.

Presently, miniature accessories are now commercially available for dolls and toy figures such as miniature infant seat, child car seat, child car bed, stroller, infant bed, hammock, cradle, rocker, bassinet, shopping cart, infant carrier, baby carrying pack, swing, crib, walker, baby jogger, high chair, stationary infant stand and various other devices appropriate to the range of activities entailed in rearing a doll.

The dolls may have the shape of a baby, animal, clown and the like, and the dolls are placed in these accessory means in a laid or seated position.

Each of these accessory means for dolls also has a bottom seat portion that supports the thighs and/or pelvis part of a doll (an occupant) and backrest portion that is adapted to provide support to the back part of the dolls.

When an infant is wrapped in a wrapping means such as a blanket, the upper portion and lower portion of the wrapping means touch the back and buttock of the infant, respectively. Further, when the infant wrapped in the wrapping means is seated in a chair, the upper portion and lower portion of the wrapping means are placed on the back member and the seat member of the chair, respectively.

If an infant is laid down on the flat surface of the bed portion of a crib, the lower portion and upper portion of the bed to which the buttock and the spine of the infant touches, respectively, is designated herein as the “bottom seat potion” and the “backrest portion”, respectively, for convenience.

Thus, for convenience, the terms “seat member” and “back member” are representatively used to designate the “bottom seat portion” and “backrest portion” of the baby holding means, accessory means and wrapping means, respectively, throughout the specification.

Also for convenience, the term “sitting means” is sometimes used to representatively designate the “baby holding means”, “accessory means” and “wrapping means” throughout the specification.

Further, also for convenience, the term “chair” or “seat” is sometimes used to representatively designate all these types of sitting means.

In a broad way, the right sitting posture in a chair is to let the hip closely contact the lower part of the back member and to let the spinal column be so physiologic, erect and static as to maintain the biomechanical, neutral spine position of a person having a normal posture in which muscular balance is maintained.

FIGS. 1 and 2 illustrate the right and faulty sitting postures of a person sitting on the work chair and driving seat, respectively.

FIGS. 3 and 4 schematically depict the pertinent skeletal components of the person shown in the left and right of FIG. 2 in phantom, respectively.

It is well known that faulty sitting posture in the chair is the most common cause of lowback pain and sciatica. Especially, in case of babies and children who grow rapidly, it is of great importance for them to have the habit to sit in right sitting posture.

Childhood play is not aimless, but represents the earliest form of apprenticeship. The process of childhood development involves imitative behavior in which the playing child carries out an action that he has been repeatedly heard and/or taught. Thus, if a child has been repeatedly heard and/or taught to take right sitting posture, he thereby gains at least a naive understanding regarding the nature of good sitting posture.

In case of a child car seat, it is provided with a shield and/or safety belts that protect the child in case the car is involved in an accident or stops abruptly.

However, children will under some conditions be sleepy and slide downwardly in the seat, tilt sideways, or to an unnatural, posture-poor position, while others, normally active children, tend to stand in these car seats, or even intentionally to climb out of them.

Moreover, it is often difficult to determine if the safety belts are securely fastened or whether any activity by the child may have caused their latching mechanism to open.

If the baby stands up abruptly or sneakily slipped out of the child car seat, the mother who is concentrating on her driving maybe quite frightened and disturbed in her driving, which may cause emergency stop, unexpected accident, or collision.

If a child has been repeatedly heard and/or taught to let the dolls in a miniature car seat take right sitting posture or to buckle the safety belts, he thereby gains at least a naive understanding regarding the nature of good sitting posture and why it is important to buckle the seat belts.

Normally, a sleeping child is laid on a wrapping means (blanket, pad, mattress and the like) and the mother is doing other things while the child is sleeping.

However, while sleeping, the baby tosses and turns, moves about and is displaced significantly from the intended position in the wrapping means, which may sometimes result in an unexpected accident.

Thus, in order to teach the concept of right sitting posture to the child and notify the parents of the displacement of the infant, baby or child from the intended position on the blanket or in the child car seat, above said various embodiments of the sensing device of the present invention are provided in the child holding means, accessory means and wrapping means of various types. By the same token, the sensing device of the present invention is also embodied in the bodies of the dolls and toy figures and the clothes for the infants, babies and children including diapers.

### DESCRIPTION OF THE PRIOR ARTS

In the prior arts, there have not been any apparatuses that notify an occupant (infant, baby or child) and/or the attendant to the occupant of the posture of the occupant, and encourage the occupant and/or the attendant to correct the posture of the occupant.

### OBJECTS OF THE INVENTION

It is therefore an object of the present invention to provide a device that notify, teach, lead or encourage an occupant in a sitting means to have optimum sitting posture or an attendant (to the occupant) to let an occupant in a sitting means have optimum sitting posture. It is a further object of the present invention to provide a sensing device in combination with various means for infant, baby and child for the infant, baby and child to have optimum posture.

The ultimate object of the present invention is to provide a device that leads a person to habitually sit in optimum sitting posture from his/her early days such that; first, he/she can have well built straight body, second, he/she can have physiologically erect posture even in his/her old age, and third, an attendant (to the occupant) can perceive the posture of the occupant and can prevent unexpected accident.

### SUMMARY OF THE INVENTION

The sensing device of the present invention basically comprises one or a plurality of sensing means, a responding means, a switch part and a microcomputer, which are electrically interconnected to one another and are provided to the part(s) of various means for the infant, baby and child to which the body of the infant, baby and child is contacting.

Said sensing means generates/sends a signal to the microcomputer when an occupant sits on the sitting means and contacts said sensing means. Upon receiving the signal, the microcomputer orders the responding means either to respond or not, according to predetermined program.

Accordingly, when the responding means responds, the occupant, upon recognizing the responding signal, tries to contact all the sensing means in order to stop the response by correcting his/her sitting posture, such that the sensing device notifies, teaches, leads and/or encourages the occupant to have optimum sitting posture.

### BRIEF DESCRIPTION OF DRAWINGS

FIGS. 1 and 2 illustrate good sitting postures and faulty sitting postures of a person sitting in a chair and a seat, respectively.

FIGS. 3 and 4 are side elevation views of a person sitting in the seat of FIG. 2, wherein the most pertinent portions of the skeletal structure of the person are depicted in phantom.

FIGS. 5 through 11 are perspective views of chairs to which the sensing device of the present invention are provided.

FIG. 12 shows in perspective a child car seat as an example of child supporting means.

FIG. 13 shows in perspective a cradle as an example of accessory means for a doll.

FIG. 14 shows the cradle in FIG. 13, which has a baby doll positioned therein and does the role of a rocker.

FIG. 15 is a plan view of a blanket as an example of child wrapping means.

FIG. 16 is a diagrammatic perspective view of the blanket in FIG. 15 placed on a child car seat.

FIGS. 17 through 20 are block diagrams of sensing devices in accordance with the present invention.

FIG. 21 is a flow chart showing a main routine for operational procedures of sensing device of the present invention. Like numerals refer to like parts throughout the several views of the drawings.

Like numerals refer to like parts throughout the several views of the drawings.

### BEST MODES FOR CARRYING OUT THE INVENTION

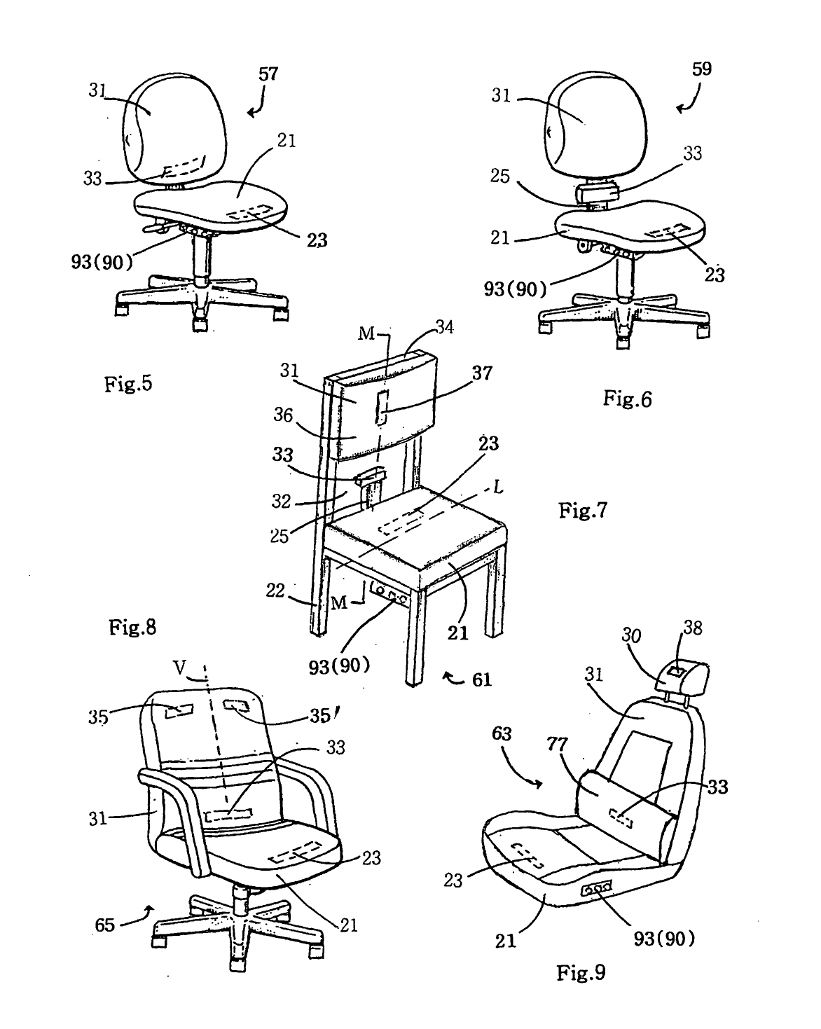

Referring to FIGS. 5 and 17, there are shown a sensing device and a chair (57) having seat member (21) and back member (31) combined with said sensing device in accordance with the present invention.

The term “sitting means” is representatively used to designate various types of means each of which defines a seat member and a back member that support the thighbones, hip, spine, ribs, shoulder blades, and/or the head of the occupant.

The sensing device basically comprises the seat sensor (SS) (23) and the hip sensor (HS) (33), a responding means (RM) (95), a switch part (SP) (93), a power supply (PS) (97) and a microcomputer (91).

The seat sensor (23) is provided to the part of the seat member (21) to which thighs and pelvis of an occupant contact and the hip sensor (33) is provided to the lower part of the back member (31) to which the hip of the occupant contacts.

The unit or assembly comprised of microcomputer (91), switch part (93), responding means (95) and power supply (97) is designated as a “control unit (90)”.

The operating mode of the sensing device of the present embodiment comprised of the seat sensor (23), hip sensor (33) and the control unit (90) is designated herein as “hip-sensing mode”.

The sensor may be any sensor (i.e. electrical, mechanical, piezoelectric, thermal, infrared LED, silicon rubber sensor, and so on) that generates electrical signal when a physical body contacts thereto, an external force is applied thereto or the sensor is activated upon detecting the presence of a physical body, which is well known to those skilled in the art.

When an occupant sits on the sitting means and contacts or activates the sensors provided to the sitting means (e.g. sensors (23), (33)), each sensor generates an electrical signal and sends it to the microcomputer (91).

The microcomputer (91) stores in memory data for the sensor(s) provided to the sitting means, data for the signal generated by each sensor, and data for the number(s) of said sensor(s), in a predetermined program, and the microcomputer (91) is further programmed to discriminate which signal is generated by which sensor, to compare the number of the signal(s) sent by the sensor(s) with the number of the sensor(s) preset in said predetermined program, and to order the responding means either to respond or not, according to said predetermined program.

Thus, the microcomputer (91) operates according to the predetermined program as follows. First, the microcomputer (91) examines the presence of signal(s) sent thereto by the sensor(s). If no signal is detected, the microcomputer (91) let the responding means (95) remain in non-responding state. If there exist a signal(s) sent thereto, the microcomputer (91) discriminates which signal is generated by which sensor and compares the number of the signal(s) with the preset number of the sensor(s). Then, the microcomputer (91) orders the responding means (95) either to respond or not, according to the predetermined program.

In detail, when an occupant sitting on the sitting means contacts the sensor(s), and thereby the number of the signal(s) sent to the microcomputer (91) coincides with the preset number of the sensor(s), the microcomputer (91) let the responding means (95) remain in non-responding state. And, when an occupant sitting on the sitting means does not contact at least any one sensor, and thereby the number of the signal(s) sent to the microcomputer (91) does not coincide with the preset number of the sensor(s), the microcomputer (91) orders the responding means (95) to produce responding signal.

When the responding means (95) produces responding signal, it is arranged such that the response is stopped when the occupant, upon recognizing the responding signal, corrects his/her sitting posture and contacts all the sensor(s) the number of which is preset in the microcomputer (91).

The responding means (RM) (95) includes devices for producing an auditory signal such as an electronic tone generator or a speaker, devices for producing a visible signal such as a light bulb or a light-emitting diode (LED) and/or devices for producing a vibratory signal such as a mechanical vibrator unit, known in the art.

The responding means (95) produces an auditory signal (sound), a visible signal (light) and/or a vibratory signal (vibration) in accordance with the order of said microcomputer.

The numeral (93) designates switch part (SP). The switch part (93) is provided with several switches the kinds and functions of which will be explained later. The numeral (97) designates power supply (PS). The power supply (97) may be battery supplied, for example, within the control unit (90) or A/C power supplied via a plug.

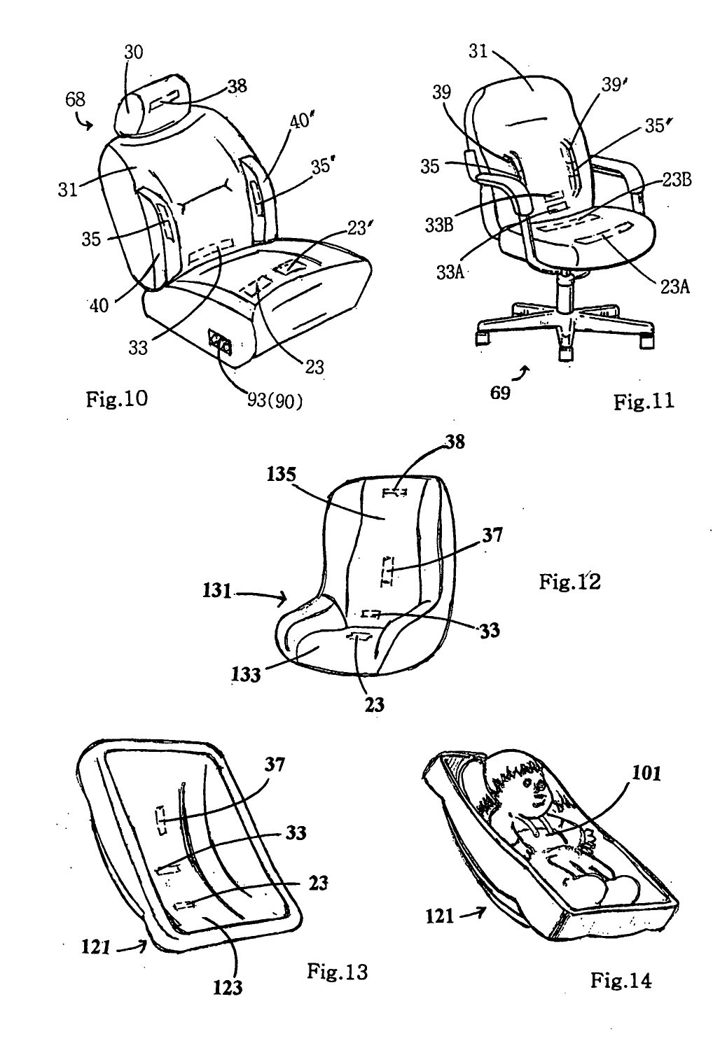

The sensors (e.g. (23) and (33)), microcomputer (91), responding means (95), switch part (93), and power supply (97) are electrically interconnected to one another by electric wires and/or circuits. The control unit (90) can be affixed to or positioned within the sitting means. In FIGS. 5, 6 and 7 the control unit (90) is affixed to the bottom side of the seat member (21) and in FIGS. 9 and 10 the control unit (90) is positioned at the lateral wall of a seat cushion (seat member (21)).

In FIGS. 5, 7, etc., the seat sensor (23) is provided to the thigh parts of the seat member (21) to which the thighbones including the ischial tuberosities contact.

The hip sensor (33) is provided to the lower part of the back member (31) on which the hip contacts when an occupant let the hip be posteriorly and inferiorly positioned to contact the lower part of the back member (31) as shown in FIGS. 5 through 16.

A child car seat such as shown in FIG. 12 generally comprises a shell of rigid plastics material molded into a rough L-shape with its raised sides curved round to form a partial enclosure. The shell is usually provided with a layer of padding material over most of its inner surface. In this case, above said sensors are provided to this padding.

The operating procedure of the hip-sensing mode is as follows.

When an occupant sits on a seat and touches the seat sensor (23) and hip sensor (33), each sensor generates and sends a signal to the microcomputer (91), respectively.

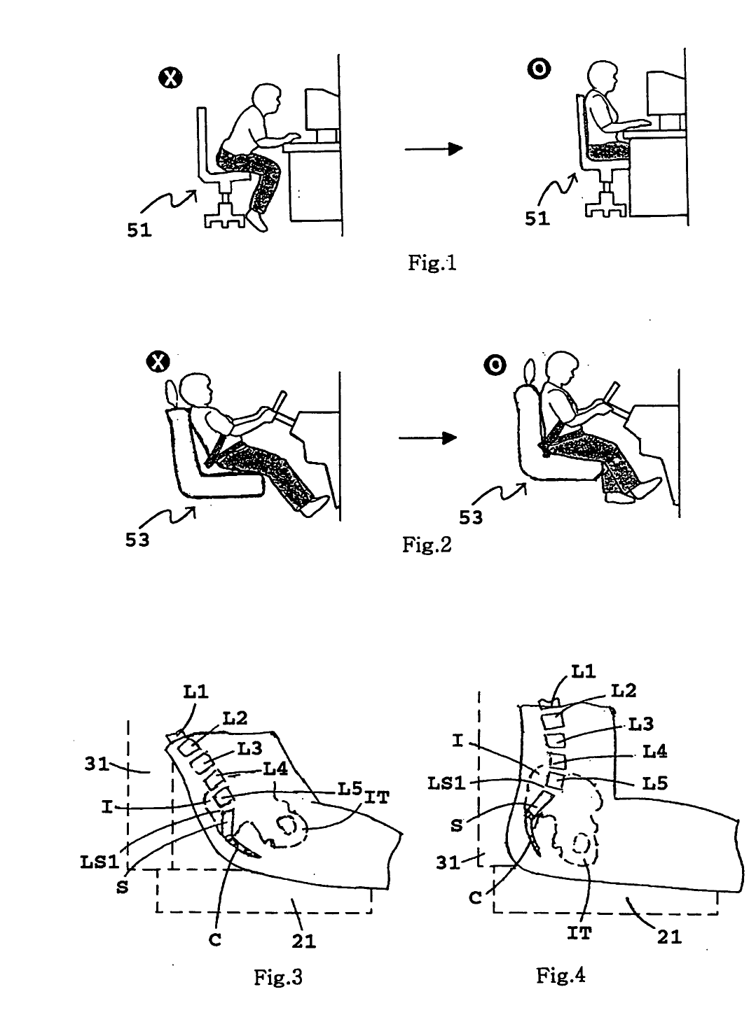

When an occupant sits in right sitting posture, in a similar fashion shown in the right of FIGS. 1 and 2, with the thighbones contacting the seat sensor (23) and the hip contacting the hip sensor (33), the two signals generated by the sensors (23) and (33) are sent to the microcomputer (91). Upon receiving all the signals of the sensors (23) and (33), the microcomputer (91) let the responding means (95) remain in non-responding state.

When an occupant sits in faulty sitting posture in a similar manner shown in the left of FIGS. 1 and 2, he/she merely contacts anyone of the sensors (23) and (33). Accordingly, the untouched sensor does not generate/send a signal to the microcomputer (91).

Upon receiving the signal the number of which is less than the preset number of the sensors, the microcomputer (91) orders the responding means (95) to produce responding signal (such as sound, light and/or vibration). Thus, when the responding means produces responding signal, the occupant becomes aware that he/she does not contact the sensor (23) and/or the sensor (33).

Immediately after the response, the occupant tries to have right sitting posture with the thigh bones contacting the seat sensor (23) and the hip contacting the hip sensor (33) by letting the hip be posteriorly and inferiorly positioned and contact the lower part of the back member (31). Thus, as the signals of all the sensors (23) and (33) are sent to the microcomputer (91), the microcomputer (91) orders the responding means (95) to stop producing responding signal.

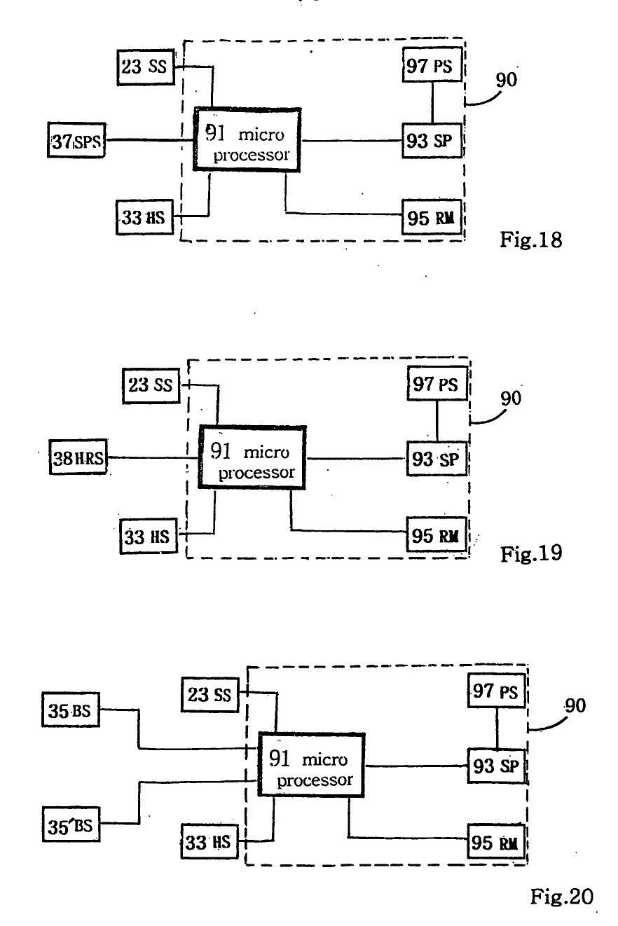

A further sensor(s) may be provided to the sitting means. In FIGS. 7, 13 and 18, a spine sensor (SPS) (37) is provided to the middle part of the back member (31) of a chair (61), along the vertical line (V) at the center of the back member (31), to which the thoracic vertebrae contact when the occupant straightens his/her spinal column. The operating mode comprised of the seat sensor (23), hip sensor (33), spine sensor (37) and the control unit (90) is designated herein as “spine-sensing mode”.

In FIGS. 8 and 20, the blade sensors (BLS) (35), (35′) are provided to the upper part of the back member (31) of a chair (65) to which the shoulder blades and/or ribs are contacting when the occupant straightens his/her spinal column and expands the chest.

Further, as shown in FIG. 11, the back member (31) of a chair 69 has a pair of slightly raised portions (39), (39′) on the surface thereof in order to fit the natural physical curvature of the ribs of an occupant and the blade sensors (35) and (35′) are provided to said raised portions, respectively.

In some automobile seats similar to the seat (68) shown in FIG. 10, there has been available a pair of side support members (40), (40′) at the respective lateral sides of the back member (31) in order to support both sides (ribs) of an occupant on the seat. A child car seat such as shown in FIG. 12 generally has raised sides curved round to form a partial enclosure.

The blade sensors (35) and (35′) may be provided to these side support members (40), (40′) or the raised side portions of a child car seat to contact the ribs of an occupant.

The operating mode comprised of the seat sensor (23), hip sensor (33), blade sensors (35), (35′) and the control unit (90) is designated herein as “blade-sensing mode”.

In FIGS. 9 and 19, a head sensor (HRS) (38) is provided to the middle part of the headrest (30) of the back member (31) of an office chair (63), to which the cranial bone of an occupant contacts when the occupant rests his/her head thereon.

The head sensor (38) is provided to prevent the unconscious tilting of the head of the occupant and to improve the resting posture of the head.

The operating mode comprised of the seat sensor (23), hip sensor (33), head sensor (38) and the control unit (90) is designated herein as a “head-sensing mode”.

In case of a car seat, restraining means such as safety belt(s) and/or shield(s) is provided to the seat to secure the safety of the occupant. In this case, a sensor(s) may be provided to these restraining means (not shown).

When the sensor(s) of the present invention is provided to the restraining means, it not only helps the occupant to sit in right sitting posture but also reminds the occupant of buckling of the restraining means.

The sensor provided to the restraining means is designated as the “belt sensor”. The operating mode comprised of the seat sensor 23 and/or hip sensor 33, belt sensor and the control unit 90 is designated herein as “belt sensing mode”.

The sensor(s) may be provided only to the back member (31).

Fist, only the hip sensor (33) may be provided to the lower part of the back member (31). The operating mode of the sensing device comprised of the hip sensor (33) and the control unit (90) is designated herein as a “basic sensing mode”. The operating procedure of the basic sensing mode will be described later.

A plurality of sensors basically including the hip sensor (33) may be provided only to the back member (31). The operating mode comprised of a plural ity of sensors provided to the back member (31) and the control unit (90) is designated herein as a “back-sensing mode”. If desired, all the sensors for the sensing modes heretofore described may altogether be provided to one sitting means. Further, in addition to these sensor(s), a certain sensor(s) may be provided to a certain place(s) of the seat member and/or back member for certain purposes. For example, a further sensor may be provided to the right or left upper part of the back member for the patient having scoliosis.

Still a further embodiment of the present invention relates to the sensing devices of the present invention combined with baby holding means.

Generally, a baby holding means is designed for providing support and/or substantial protection of the occupant (an infant, baby or child) and capable of being oriented in either a sitting and/or reclining position.

As above described, the baby holding means designates any one of a number of such devices as an infant seat, child car seat, child car bed, stroller, infant bed, hammock, cradle, rocker, bassinet, shopping cart, infant carrier, baby carrying pack, swing, crib, walker, baby jogger, high chair, stationary infant stand and the like. Each of the baby holding means is provided as a separate unit and some are designed to function as a combination of a few of these devices.

Each of the baby holding means comprises substantially flat bottom seat portion and backrest portion that receive and/or support the body of the occupant.

Further, the bottom seat portion and backrest portion have thigh parts, hip part, spine part, shoulder blade parts and/or the head part to which the thighs, hip, spine, shoulder blades and/or the head of an occupant contact, respectively.

The sensors in the above embodiments are provided to the thigh parts, hip part, spine part, shoulder blade parts and/or the head part of the bottom seat portion and the backrest portion.

For example, in FIG. 12, seat sensor (23), hip sensor (33), spine sensor (37) and head sensor (38) are provided to the thigh parts, hip part, spine part and the head part of the bottom seat portion (133) and backrest portion (135) of the baby holding means (131) (child car seat). The control unit (90) is connected to these sensors through electric wires (not shown). When a hood is provided to these baby holding means such as a bunting, the head sensor (38) is provided to the middle head part of the hood.

Still a further embodiment of the present invention relates to the sensing devices of the present invention combined with accessory means for dolls.

Generally, an accessory means is designed for providing support to the occupant (a doll) that is placed therein in a sitting and/or reclining position.

As above described, the accessory means for dolls designates any one of a number of such devices as a miniature of infant seat, child car seat, child car bed, stroller, infant bed, hammock, cradle, rocker, bassinet, shopping cart, infant carrier, baby carrying pack, swing, crib, walker, baby jogger, high chair, stationary infant stand and various other devices that hold, carry or support dolls.

Each of the accessory means also has a substantially flat bottom seat portion and backrest portion that receive and/or support the body of the occupant (a toy means such as a doll).

Further, these bottom seat portion and backrest portion also have thigh parts, hip part, spine part, shoulder blade parts and/or the head part to which the thighs, hip, spine, shoulder blades and/or the head of an occupant contact, respectively.

The sensors in the above embodiments are also provided to the thigh parts, hip part, spine part, shoulder blade parts and/or the head part of the bottom seat portion and the backrest portion of the accessory means.

FIG. 13 shows a generally rectangular rocker cradle (121) molded of synthetic plastic material. The cradle (121) is provided with a bellied-out bottom wall (123) to receive a doll and the sensors ((23), (33) and (37)) are provided to the thigh parts, hip part and the spine part of the bottom wall (123).

In FIG. 14, the cradle (121) functions as a rocker with respect to a baby doll (101) placed in the bellied-out bottom wall (123) touching all the sensors (23), (33) and (37).

Still a further embodiment of the present invention relates to the sensing devices of the present invention combined with wrapping means (blankets and similar articles) for an infant, baby or a child.

Generally, a wrapping means is designed for providing support, warmth and/or substantial protection of the occupant such as an infant, baby or child, and is used with a child car seat or baby carrier as well as indoors.

A wrapping means generally designates any one of a substantially flat blanket, cushion support, pad, bunting or sheet article of flexible fabric material such as cotton, nylon, fleece, or the like and has a front side, back side, a contiguous upper, bottom, right and left edge and dimensions sufficient to fit around a child.

Also some wrapping means include pockets or attachments that permit the mounting of a cushion support or pad to a primary blanket. Some wraps in the markets convert to a blanket, a bunting or a pad.

When an infant is wrapped in a wrapping means and is in a laid position or seated position, the wrapping means defines a bottom seat portion and backrest portion that receive and/or support the body of the infant.

Further, the bottom seat portion and backrest portion also defines thigh parts, hip part, spine part, shoulder blade parts and/or the head part to which the thighs, hip, spine, shoulder blades and/or the head of an occupant contact, respectively.

The sensors in the above embodiments are provided to the thigh parts, hip part, spine part, shoulder blade parts and/or the head part of the bottom seat port ion and the backrest portion of the wrapping means.

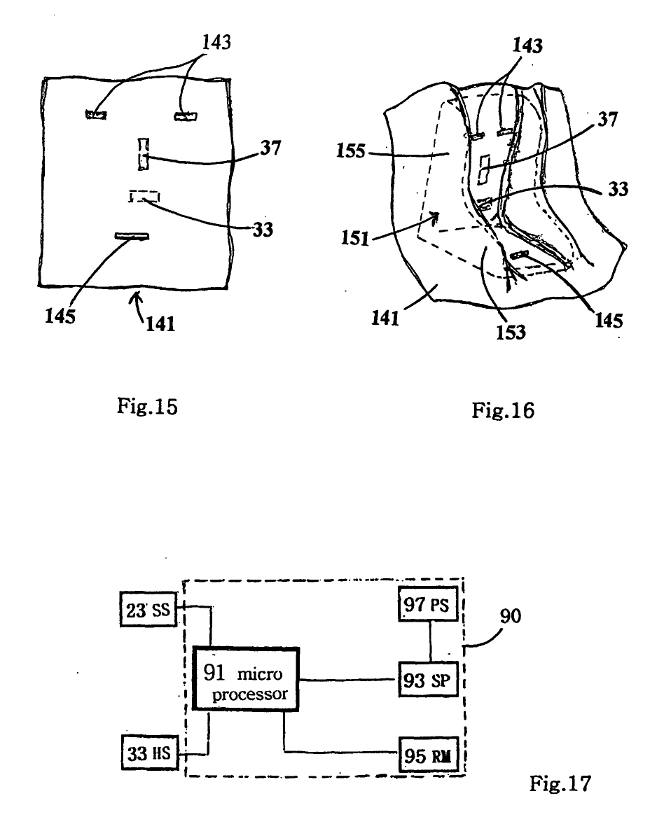

For example, in FIG. 15, hip sensor (33) and spine sensor (37) are provided to the hip part and spine part of the bottom seat portion and backrest portion of the blanket (141), respectively. The control unit (90) is connected to these sensors through electric wires (not shown).

FIG. 16 shows a wrapping means (blanket) (141) of the present embodiment placed over a child car seat (151). The numerals (143) and (145) designate the apertures to receive the safety belt and the numerals (153) and (155) designate the bottom seat portion and backrest portion of the child car seat (151), respectively.

Right on the surfaces of the bottom seat portion (153) and the backrest portion (155) of the child car seat (151) are placed the bottom seat portion and the backrest portion of the wrapping means (141), respectively.

Still a further embodiment of the present invention relates to the sensing devices of the present invention combined with the clothing means for the infants, babies and children (not shown).

Generally, a clothing means is designed for providing support, warmth and/or substantial protection of the wearer such as an infant, baby or child.

The clothing means herein designates any one of the diapers, covers for diapers, shirts, unitary garments, pants, sanitary napkins, bandages and the like for the infants, babies or children. When a clothing means is provided to an infant, the clothing means defines stomach part, thigh parts, hip part, spine part, shoulder blade parts and/or the head part to which the stomach, thighs, hip, spine, shoulder blades and/or the head of the infant contact, respectively.

The sensors in the above embodiments are provided to the stomach part, thigh parts, hip part, spine part, shoulder blade parts and/or the head part of the clothing means.

The sensor(s) may be embedded in the desired part(s) of the clothing means or affixed to said part(s) by means of the Velcro RTM fasteners.

If an infant wearing a diaper having a sensor provided to the hip part of the cover for a diaper is displaced from the bed, the sensor pressed under the hip is released and generates signal. Thus, the responding signal notifies the mother who is not present in the room that the infant has awakened and is moving about.

Still a further embodiment of the present invention relates to the sensing devices of the present invention combined with the toy means for the babies and children (not shown).

The dolls and toy figures have been provided in human resembling figures such as warriors, heroes or the like, fantasy-type figures including cartoon and other fictional characters such as a ghost or fairy godmother or other recognizable character as well as animal-like figures that generally resemble fanciful animals such as a dog, a cat, a rabbit, a bear, or the like.

Almost all of these toy means have been made of cloths, woods, rubbers, synthetic materials and/or the combination of a few of these materials.

The toy means herein designates any one of the dolls and toy figures provided in human resembling figures, fantasy-type figures, or animal-like figures for the babies or children.

Each of these toy means has stomach part, thigh parts, hip part, spine part, shoulder blade parts and/or the head part.

The sensors in the above embodiments are provided to these stomach part, thigh parts, hip part, spine part, shoulder blade parts and/or the head part of the toy means.

The sensor(s) may be embedded in the desired part(s) of the toy means or affixed to said part(s) by means of known fixing means such as the Velcro RTM fasteners, adhesives and the like.

If a doll seated in a miniature chair with its hip part having a sensor provided thereto is bent forward by an external force, the hip sensor contacting the lower part of the back member of the chair is released and generates signal.

Thus, the responding signal notifies the attendant (e.g., a child) that the doll is displaced from the intended position.

The operating procedure of above said sensing mode(s) having a plurality of sensors is similar to those described in connection with above described hip-sensing mode. Namely, when an occupant on the sitting means contacts all the sensors provided thereto, the microcomputer (91) let the responding means (95) remain in non-responding state.

When the occupant does not contact at least any one of the sensors, the microcomputer (91) orders the responding means to respond, and the microcomputer (91) orders the responding means (95) to stop producing responding signal when the occupant corrects his/her sitting posture and again contacts all the sensors.

A sensor-selecting switch may optionally be provided to the switch part (93) for the occupant to select the sensor(s) that he/she wants to operate among the sensors provided to the sitting means.

The group of sensor(s) selected by the sensor-selecting switch includes at least the hip sensor (33), and may be classified as follows.
1. hip sensor (33) 2. seat sensor (23) and hip sensor
3. seat sensor, hip sensor and spine sensor (37)
4. seat sensor, hip sensor and blade sensors (35), (35)
5. seat sensor, hip sensor and head sensor (38)
6. seat sensor, hip sensor, blade sensors and head sensor
7. hip sensor and spine sensor
8. hip sensor and blade sensors
9. hip sensor, blade sensors and head sensor, and so on.

Further, when a belt sensor(s) is provided to a sitting means that is equipped with seat belt(s), the belt sensor(s) may optionally be added to these groups.

The sensors of the present invention may be secured in or beneath the outer covering or strap(s) of cloths, leathers, vinyl or any other synthetic materials of said seat member, back member or seat belt(s). Further, the sensors may be attached on the surfaces of the seat member and back member. The sensors may be so provided in a slightly convex fashion on the surfaces of the seat member (21) and back member (31) that the body of the occupant may easily contact the sensors.

The switch part (93) is provided with several kinds of switches. The first switch may be a power supply switch for selecting on/off mode of the power supply (97). The second switch may be a responding-device-selecting switch that is capable of switching over among above said responding devices for producing an auditory signal, a visible signal and/or a vibratory signal. The third switch may be a sensor-selecting switch that enables the occupant to choose the sensor(s) he/she wants. The fourth switch may be a time adjusting switch that adjusts the standard hold time to defer the response of the responding means (95).

Further, the sensors may be comprised of weight detecting sensors that can detect the weight or pressing force of the occupant applied to the sensors.

First, a standard degree of pressing force for each sensor is preset and stored in memory.

Second, the pressing force applied to each sensor is checked through a determination as to whether the pressing force exceeds the preset standard degree or not.

Third, when the pressing force applied to a sensor (e.g. belt sensor) exceeds the standard degree, the microcomputer (91) orders the responding means (95) to produce responding signal.

Fourth, when the responding means (95) produces responding signal, it is arranged such that the response is stopped when the occupant or the attendant (to the occupant), upon recognizing the responding signal, reduces the pressing force applied to the sensor (of the seat belt by loosening the seat belt).

With these arrangements, in case of a child car seat, the attendant (to the baby) can aware whether the seat belt(s) is buckled too tight or not and can adjust the seat belt.

Further, the present invention may be of such arrangement that the microcomputer (91) maintains the responding means (95) in hold state for a short time, as a hold time, until certain standard time elapses, according to the predetermined program.

In short, the microcomputer (91) may be so programmed as to order the responding means (95) to remain in hold state for a given standard time, and after lapse of such hold time, the microcomputer (91) permits the responding means (95) to respond. A known time counting means, i.e., a timer (not shown) is provided for counting said standard hold time. The hold time may be approx. 3 sec., preferably. The standard hold time may be altered to other desired time period such as 1 sec., 5 sec., 60 sec., and so forth.

With these arrangements, it is possible to prevent the responding means to randomly generate responding signals when there exists certain time period for the occupant to contact all the corresponding sensors for certain sensing mode.

Now, with above explained sensing device of the present invention, the procedure for sensing and notifying the posture of an occupant will be described.

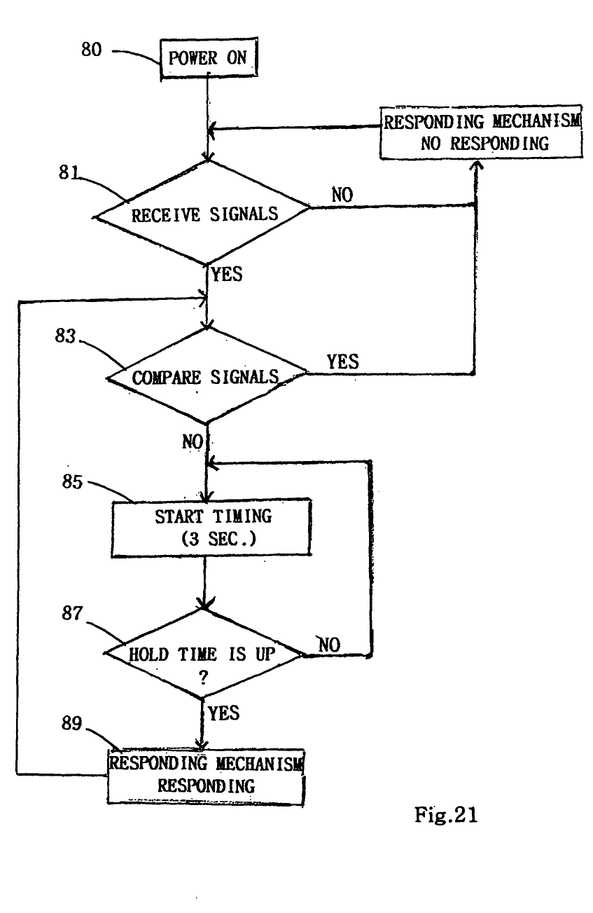

In FIG. 21, there is illustrated a main flow diagram of the processing procedures of the microcomputer (91). According thereto, at the block (80), the power supply switch is turned on and the flow of the processing step starts, as indicated by the next block (81), with an instruction to detect the presence of the signals generated by the sensors and sent to the microcomputer (91).

At the block (81), it is examined whether signals transmitted from the sensors exist or not, and determined either to put the responding means in non-responding state when there exists no signal sent by the sensors, or to let the step proceed to the next block (83) when there exists at least one signal.

It is noted here that normally, no signal is generated/sent to the microcomputer (91) when no one is sitting on the sitting means. Likewise, when the occupant sitting on the sitting means and contacting all the corresponding sensors for certain sensing mode stands up and does not contact any sensors, no signal is generated/sent to the microcomputer (91).

If no signal is detected, the “NO” is decided, thus putting the responding means (95) at the block (82) in non-responding state.

If, however, there exist at least one signal received, the block (81) answers “YES”, leading the step to the next block (83).

As described earlier, the number of sensors for certain sensing mode and the data for the signals of the sensors are preset and stored in memory for the microcomputer (91) to proceed with the operations.

At the block (83), the step proceeds to compare the number of the signal(s) sent by the sensor(s) with the preset number of corresponding sensors for certain sensing mode, and to determine as to whether the number of signals coincides with the preset number of sensors or not.

If the number of signals coincides with the preset number of the sensors, the “YES” is decided, thus putting the responding means (95) at the block (82) in non-responding state. If the occupant does not contact any one sensor(s) or contacts it (them) a little late, as the number of signals does not coincide with the preset number of the sensors, the block (83) answers “NO”, leading the step to the next block (85). At the block (85), a timer (not shown) starts to count the preset 3 seconds of hold time.

The step is then led to determining at the block (87) as to whether the 3 seconds of hold time has lapsed, or not. At this stage, during such hold time, instruction in the microcomputer (91) repeatedly indicates a “NO” signal until the 3-sec. time period is over, to withhold decision.

Then, after lapse of the hold time, “YES” is decided at the block (87), so that, at block (87), instruction is given to cause said responding means at the block (89) to respond. During the hold time, if the occupant touches the sensor(s) that he/she at first did not touch, all the signals are sent to the block (81).

Accordingly, at the block (83), the number of the signals becomes coincident with the preset number of the sensors, and the “YES” is decided at the block (83), thus stopping the counting and putting the responding means (95) at the block (82) in non-responding state.

When the hold time has lapsed and the responding means (95) begins to produce responding signal, and if the occupant, upon apprehending the response of the responding means (95), contacts the sensor(s) that he/she at first did not contact, all the signals are sent to the block (81), and thereupon the number of the signals becomes coincident with the preset number of the sensors, the “YES” is decided at the block (83), thus stopping the response of the responding means (95) and putting the responding means (95) at the block (82) in non-responding state.

As above described, the occupant contacting all the corresponding sensors for certain sensing mode may feel necessary to remove certain part of the body from the part of the sitting means to which said part of the body is contacting for a short time period, and thereupon does not touch the sensor provided to said part of the sitting means.

In this case, the number of signals sent to the block (81) is less than the preset number of the sensors, and the flow of processing step again proceeds through the blocks (83), (85), (87) in the same fashion as described above.

In case of the above described “basic sensing mode” comprised of the hip sensor (33) and control unit (90), as only the hip sensor (33) is provided to the sitting means or selected among the sensors provided to the sitting means, the microcomputer (91) is programmed to include the following two different steps of operations.

When an occupant sits in the sitting means and contacts the hip sensor (33), the sensor (33) generates and sends signal to the microcomputer (91). Upon receiving the signal the number of which coincides with the preset number of the sensor (33), the microcomputer (91) let the responding means (95) remain in non-responding-state in the same manner as described in connection with other sensing modes.

When the occupant removes the hip from the sensor (33), the sensor (33) stops generating/sending signal to the microcomputer (91), which, in the other sensing modes, means that as no signal is sent to the microcomputer (91), the microcomputer (91) puts the responding means (95) in non-responding state.

However, as the first different step of operation, when the sensor (33) stops sending signal for the first time, the microcomputer (91), even though there exists no signal sent thereto, orders the responding means (95) to produce responding signal to notify the occupant that he/she is not contacting the sensor (33).

When said responding means produces the responding signal, the occupant becomes aware that he/she does not contact said sensors. Immediately after the response, the occupant, upon recognizing the responding signal, corrects his/her sitting posture with the hip contacting the lower part of said back member by letting the hip be posteriorly and inferiorly positioned, and contacts said hip sensor within certain standard time period.

Thus, the signal of the sensor (33) is again sent to the microcomputer (91), and thereby the responding means (95) stops producing responding signal.

As the second different step of operation, if the occupant continuously does not contact the hip sensor (33) and thereby no signal is sent to the microcomputer (91) during a given standard time period, the microcomputer orders the responding means (95) to remain in non-responding state after the standard time period has elapsed.

With all these embodiments of the sensing devices, the present invention notifies the occupant and/or the attendant (to the occupant) of the posture of the occupant, and thereby aids in maintaining the occupant in a proper, posture-correct seating position, and especially, it is extremely helpful in caring for, teaching and transporting an infant, baby or child, and is of great relief to the parent in any case.

While having described the present invention thus far, it should be understood that the invention is not limited to the illustrated embodiments, but other various modifications, alterations and additions may be possible without departing from the spirits and scope of the appended claims.

1. A sensing device for a baby holding means, an accessory means for toy means, a wrapping means, a clothing means or a toy means, for notifying the posture of an occupant placed in a baby holding means, an accessory means or a wrapping means, or the posture of an wearer of a clothing means, or the posture of a toy means, to the occupant or the wearer and/or an attendant (to the occupant, the wearer or the toy means), and thereby encouraging the occupant, the wearer and/or the attendant (to the occupant, the wearer or the toy means) to correct the posture of the occupant, the wearer or the toy means,
each of said baby holding means, accessory means or wrapping means being designed for providing support, warmth and/or substantial protection of the occupant, and having or defining a flat bottom seat portion and a flat backrest portion that receive and/or support the body of the occupant,
said bottom seat portion and backrest portion defining thigh parts, hip parts, spine part, shoulder blade parts and/or the head part to which the thighs, hip, spine, shoulder blades and/or the head of an occupant contact, respectively,
said clothing means being designed for providing warmth and/or substantial protection of the wearer, and having stomach part, thigh parts, hip part, spine part, shoulder blade parts and/or the head part to which the stomach, thighs, hip, spine, shoulder blades and/or the head of the wearer contact, respectively,
said toy means being designed for providing amusement and comfort of the attendant (e.g., babies or children) and being any one of the dolls and toy figures, and having stomach part, thigh parts, hip part, spine part, shoulder blade parts and/or the head part to which an external thing contacts,
said sensing device comprising:
one or a plurality of sensors for generating electrical signal and sending said signal to a microcomputer when an occupant, a wearer or an external thing contacts or activates the sensor(s), said sensor(s) being provided to the thigh parts, hip part, spine part, shoulder blade parts, and/or the head part to which the body of the occupant or the wearer, or an external thing contacts,
a microcomputer that stores in memory data for the sensor(s), data for the signal generated by each sensor, and data for the number(s) of said sensor(s), in a predetermined program, and is further programmed to discriminate which signal is generated by which sensor, to compare the number of the signal(s) sent by the sensor(s) with the number of the sensor(s) preset in said predetermined program, and to order the responding means either to respond or not,
a responding means for producing responding signal in accordance with the order of the microcomputer, said responding means including devices for producing an auditory signal, a visible signal and/or a vibratory signal,
a switch part being provided with several kinds of switches,
a power supply being battery supplied or A/C power supplied,
said one or a plurality of sensors, microcomputer, responding means, switch part and power supply being electrically interconnected to one another, and
said microcomputer being so programmed, according to said predetermined program, that;
the microcomputer examines the presence of a signal(s) sent thereto by the sensor(s), and if no signal is detected, the microcomputer let the responding means remain in non-responding state,
when an occupant, a wearer or an external thing contacts the sensor(s) provided to said baby holding means, accessory means, wrapping means, clothing means or toy means, and the number of the signal(s) sent to the microcomputer coincides with the preset number of the sensor(s), the microcomputer orders the responding means to remain in non-responding state,
when the occupant, the wearer or an external thing does not contact or activate at least any one sensor and thereby the number of the signal(s) sent to the microcomputer is less than the preset number of the sensor(s), the microcomputer orders the responding means to produce responding signal, and
it is arranged such that the response is stopped when the occupant, the wearer or the attendant (to the occupant, the wearer or the toy means), upon recognizing the responding signal, corrects the posture of the occupant, the wearer or the toy means,
and thereby let all the sensors the number of which is coincident with the preset number of the sensor(s) be contacted by the occupant, the wearer or an external thing.

2. A combination of a sensing device with a baby holding means for notifying an occupant placed in the baby holding means and/or an attendant (to the occupant) of the posture of the occupant, and thereby encouraging the occupant and/or the attendant to correct the posture of the occupant, comprising:
a baby holding means being designed for providing support and/or protection of the occupant and capable of being oriented in either a sitting and reclining position, and having a bottom seat portion and backrest portion that, respectively, have substantially flat surfaces and adequate sizes to receive and/or support the body of the occupant,
said bottom seat portion and backrest portion defining thigh parts, hip part, spine part, shoulder blade parts and/or the head part to which the thighs, hip, spine, shoulder blades and/or the head of an occupant contact, respectively, and
a sensing device for said baby holding means:
said sensing device comprising;
one or a plurality of sensors for generating electrical signal when an occupant contacts or activates the sensor(s) and sending said signal to a microcomputer, said sensor(s) being provided to the thigh parts, hip part, spine part, shoulder blade parts and/or the head part of said bottom seat portion and backrest portion,
a microcomputer that stores in memory data for the sensor(s), data for the signal generated by each sensor, and data for the number(s) of said sensors(s), in a predetermined program, and is further programmed to discriminate which signal is generated by which sensor, to compare the number of the signal(s) sent by the sensor(s) with the number of the sensor(s) preset in the predetermined program, and to order the responding means either to respond or not,
a responding means for producing responding signal in accordance with the order of the microcomputer, said responding means including devices for producing an auditory signal, a visible signal and/or a vibratory signal,
a switch part being provided with several kinds of switches,
a power supply being battery supplied or A/C power supplied,
said one or a plurality of sensors, microcomputer, responding means, switch part and power supply being electrically interconnected to one another and provided to said baby holding means; and
said microcomputer being so programmed, according to said predetermined program, that;
the microcomputer examines the presence of a signal(s) sent thereto by the sensor(s), and if no signal is detected, the microcomputer let the responding means remain in non-responding state,
when an occupant placed on the baby holding means contacts the sensor(s) and the number of the signal(s) sent to the microcomputer coincides with the preset number of the sensor(s) the microcomputer orders the responding means to remain in non-responding state,
when the occupant does not contact or activate at least any one sensor and thereby the number of the signal(s) sent to the microcomputer is less than the preset number of the sensor(s), the microcomputer orders the responding means to produce responding signal, and
it is arranged such that the response is stopped when the occupant and/or the attendant (to the occupant), upon recognizing the responding signal, corrects the posture of the occupant, and thereby let the occupant contact or activate all the sensor(s) the number of which is coincident with the preset number of the sensor(s).

3. A combination of a sensing device with an accessory means for a toy means for notifying an attendant to the occupant (e.g., a doll) placed in the accessory means of the posture of the occupant, and thereby encouraging the attendant to correct the posture of the occupant, comprising:
an accessory means being designed for providing support and/or substantially protection of the occupant and capable of being oriented in either a sitting and reclining position, and having a bottom seat portion and backrest portion that, respectively, have substantially flat surfaces and adequate sizes to receive and/or support the body of the occupant, and
said bottom seat portion and backrest portion defining thigh parts, hip part, spine part, shoulder blade parts and/or the head part to which the thighs, hip, spine, shoulder blades and/or the head of an occupant (e.g., a doll) contact, respectively,
a sensing device for said accessory means:
said sensing device comprising;
one or a plurality of sensors for generating electrical signal when an occupant contacts or activates the sensor(s) and sending said signal to a microcomputer, said sensor(s) being provided to the thigh parts, hip part, spine part, shoulder blade parts and/or the head part of said bottom seat portion and backrest portion,
a microcomputer that stores in memory data for the sensor(s), data for the signal generated by each sensor, and data for the number(s) of said sensor(s), in a predetermined program, and is further programmed to discriminate which signal is generated by which sensor, to compare the number of the signal(s) sent by the sensor(s) with the number of the sensor(s) preset in the predetermined program, and to order the responding means either to respond or not,
a responding means for producing responding signal in accordance with the order of the microcomputer, said responding means including devices for producing an auditory signal, a visible signal and/or a vibratory signal,
a switch part being provided with several kinds of switches,
a power supply being battery supplied or A/C power supplied,
said one or a plurality of sensors, microcomputer, responding means, switch part and power supply being electrically interconnected to one another and provided to said accessory means; and
said microcomputer being so programmed, according to said predetermined program, that;
the microcomputer examines the presence of a signal(s) sent thereto by the sensor(s), and if no signal is detected, the microcomputer let the responding means remain in non-responding state,
when an occupant (a toy means) placed on the accessory means contacts the sensor(s) and the number of the signal(s) sent to the microcomputer coincides with the preset number of the sensor(s), the microcomputer orders the responding means to remain in non-responding state,
when the occupant does not contact or activate at least any one sensor and thereby the number of the signal(s) sent to the microcomputer is less than the preset number of the sensor(s), the microcomputer orders the responding means to produce responding signal, and
it is arranged such that the response is stopped when the attendant (to the occupant), upon recognizing the responding signal, corrects the posture of the occupant, and thereby let the occupant contact or activate all the sensor(s) the number of which is coincident with the preset number of the sensor(s).

4. A combination of a sensing device with a wrapping means for notifying an occupant placed in the wrapping means and/or an attendant to the occupant of the posture of the occupant, and thereby encouraging the occupant and/or the attendant to correct the posture of the occupant, comprising:
a wrapping means being designed for providing support, warmth and/or substantial protection of the occupant, and defining a bottom seat portion and backrest portion that, respectively, have substantially flat, flexible surfaces and adequate sizes to receive and/or support the body of the occupant, and
said bottom seat portion and backrest portion defining thigh parts, hip part, spine part, shoulder blade parts, and/or head part to which the thighs, hip, spine, shoulder blades and/or the head of the occupant contact, respectively,
a sensing device for said wrapping means:
said sensing device comprising;
one or a plurality of sensors for generating electrical signal when an occupant contacts or activates the sensor(s) and sending said signal to a microcomputer, said sensor(s) being provided to the thigh parts, hip part, spine part, shoulder blade parts and/or the head part of said bottom seat portion and backrest portion,
a microcomputer that stores in memory data for the sensor(s), data for the signal generated by each sensor, and data for the number(s) of said sensor(s), in a predetermined program, and is further programmed to discriminate which signal is generated by which sensor, to compare the number of the signal(s) sent by the sensor(s) with the number of the sensor(s) preset in the predetermined program, and to order the responding means either to respond or not,
a responding means for producing responding signal in accordance with the order of the microcomputer, said responding means including devices for producing an auditory signal, a visible signal and/or a vibratory signal,
a switch part being provided with several kinds of switches,
a power supply being battery supplied or A/C power supplied,
said one or a plurality of sensors, microcomputer, responding means, switch part and power supply being electrically interconnected to one another and provided to said wrapping means; and
said microcomputer being so programmed, according to said predetermined program, that;
the microcomputer examines the presence of a signal(s) sent thereto by the sensor(s), and if no signal is detected, the microcomputer let the responding means remain in non-responding state,
when an occupant placed in the wrapping means contacts the sensor(s) and the number of the signal(s) sent to the microcomputer coincides with the preset number of the sensor(s), the microcomputer orders the responding means to remain in non-responding state,
when the occupant does not contact or activate at lease any one sensor and thereby the number of the signal(s) sent to the microcomputer is less than the preset number of the sensor(s), the microcomputer orders the responding means to produce responding signal, and
it is arranged such that the response is stopped when the occupant and/or the attendant (to the occupant), upon recognizing the responding signal, corrects the posture of the occupant, and thereby let the occupant contact or activate all the sensor(s) the number of which is coincident with the preset number of the sensor(s).

5. A combination of a sensing device with a clothing means for notifying a wearer wearing the clothing means and/or an attendant to the wearer of the posture of the wearer, and thereby encouraging the wearer and/or the attendant to correct the posture of the wearer, comprising:
a clothing means being designed for providing warmth and/or substantial protection of the wearer, and defining stomach part, thigh parts, hip part, spine part, shoulder blade parts and/or the head part to which the stomach, thighs, hip, spine, shoulder blades and/or the head of the wearer contact, respectively, and
a sensing device for said clothing means:
said sensing device comprising;
one or a plurality of sensors for generating electrical signal when a wearer contacts or activates the sensor(s) and sending said signal to a microcomputer, said sensor(s) being provided to said stomach part, thigh parts, hip part, spine part, shoulder blade parts and/or the head part, and
a microcomputer that stores in memory data for the sensor(s), data for the signal generating by each sensor, and data for the number(s) of said sensor(s), in a predetermined program, and is further programmed to discriminate which signal is generated by which sensor, to compare the number of the signal(s) sent by the sensor(s) with the number of sensor(s) preset in the predetermined program, and to order the responding means either to respond or not,
a responding means for producing responding signal in accordance with the order of the microcomputer, said responding means including devices for producing an auditory signal, a visible signal and/or a vibratory signal,
a switch part being provided with several kinds of switches,
a power supply being battery supplied or A/C power supplied,
said one or a plurality of sensors, microcomputer, responding means, switch part and power supply being electrically interconnected to one another and provided to said clothing means; and
said microcomputer being so programmed, according to said predetermined program, that;
the microcomputer examines the presence of a signal(a) sent thereto by the sensor(s), and if no signal is detected, the microcomputer let the responding means remain in non-responding state,
when a wearer wearing the clothing means contacts the sensor(s) and thereby the number of the signal(s) sent to the microcomputer coincides with the preset number of the sensor(s), the microcomputer orders the responding means to remain in non-responding state,
when the wearer does not contact or activate at least any one sensor and thereby the number of the signal(s) sent to the microcomputer is less than the preset number of the sensor(s), the microcomputer orders the responding means to produce responding signal, and
it is arranged such that the response is stopped when the wearer and/or the attendant (to the wearer), upon recognizing the responding signal, corrects the posture of the wearer, and thereby let the wearer contact or activate all the sensor(s) the number of which is coincident with the preset number of the sensor(s).

6. A combination of a sensing device with a toy means for notifying an attendant to the toy means of the posture of the toy means, and thereby encouraging the attendant to correct the posture of the toy means, comprising:
a toy means being designed for providing amusement and comfort of the attendant (e.g., babies or children) and being any one of the dolls and toy figures provided in human resembling figures, fantasy-type figures, or animal-like figures, and having stomach part, thigh parts, hip part, spine part, shoulder blade parts and/or the head part to which an external thing contacts, and
a sensing device for said toy means:
said sensing device comprising;
one or a plurality of sensors for generating electrical signal when an external thing contacts or activates the sensor(s), said sensor(s) being provided to said stomach part, thigh parts, hip part, spine part, shoulder blade parts and/or the head part,
a microcomputer that stores in memory data for the sensor(s), data for the signal generated by each sensor, and data for the number(s) of said sensor(s), in a predetermined program, and is further programmed to discriminate which signal is generated by which sensor, to compare the number of the signal(s) sent by the sensor(s) with the number of the sensor(s) preset in the predetermined program, and to order the responding means either to respond or not,
a responding means for producing responding signal in accordance with the order of the microcomputer, said responding means including devices for producing an auditory signal, a visible signal and/or a vibratory signal,
a switch part being provided with several kinds of switches,
a power supply being battery supplied or A/C power supplied,
said one or a plurality of sensors, microcomputer, responding means, switch part and power supply being electrically interconnected to one another and provided to said toy means; and
said microcomputer being so programmed, according to said predetermined program, that;
the microcomputer examines the presence of a signal(s) sent thereto by the sensor(s), and if no signal is detected, the microcomputer let the responding means remain in non-responding state,
when the sensor(s) of the toy means placed on an external thing is contacted by the external thing and the number of the signal(s) sent to the microcomputer coincides with the preset number of the sensor(s), the microcomputer orders the responding means to remain in non-responding state,
when at least any one sensor of the toy means is not contacted by the external thing and is not activates, and thereby the number of the signal(s) sent to the microcomputer is less than the preset number of the sensor(s), the microcomputer orders the responding means to produce responding signal, and
it is arranged such that the response is stopped when the attendant (to the toy means), upon recognizing the responding signal, corrects the posture of the toy means, and thereby let the toy means contact an external thing and activate all the sensor(s) the number of which is coincident with the preset number of the sensor(s).

7. The sensing device according to claim 1, wherein said sensors comprises as follows;
a seat sensor is provided to the thigh parts of said baby holding means, accessory means, wrapping means, clothing means or toy means, and a hip sensor is provided to the hip part thereof.

8. The sensing device according to claim 1, wherein said sensors comprises as follows;
a seat sensor is provided to the thigh parts of said baby holding means, accessory means, wrapping means, clothing means or toy means, a hip sensor is provided to the hip part thereof and a spine sensor is provided to the spine part thereof.

9. The sensing device according to claim 1, wherein said sensors comprises as follows:
a seat sensor is provided to the thigh parts or said baby holding means, accessory means, wrapping means, clothing means or toy means, a hip sensor is provided to the hip part thereof and two blade sensors are provided to the shoulder blade parts thereof.

10. The sensing device according to claim 1, wherein said sensors comprises as follows;
a seat sensor is provided to the thigh parts of said baby holding means, accessory means, wrapping means, clothing means or toy means, a hip sensor is provided to the hip part thereof and a head sensor is provided to the head part thereof.

11. The sensing device according to claim 1, wherein said sensors comprises as follows;
a hip sensor is basically provided to the hip part of said baby holding means, accessory means, wrapping means, clothing means or toy means and
at lease one sensor among the spine sensor blade sensors, and a head sensor is further provided to the respective spine part, shoulder blade parts or the head part thereof.

12. The sensing device according to claim 1, wherein said switch part includes a sensor-selecting switch for an occupant, a wearer or an attendant (to the occupant, the wearer or the toy means) of said baby holding means, accessory means, wrapping means, clothing means or toy means to select the sensor(s) that he/she wants to operate.

13. The sensing device according to claim 1, wherein said sensors comprises as follows;
only a hip sensor is provided to the hip part of said baby holding means, accessory means, wrapping means, clothing means or toy means, or only the hip sensor is selected among all the sensors provided to the baby holding means, accessory means, wrapping means, clothing means or toy means by a sensor-selecting switch.

14. The sensing device according to claim 1, wherein said microcomputer is so programmed, in a predetermined program, as to maintain the responding means in hold state for a given standard time, until said standard time elapses, and after lapse of such standard hold time, the microcomputer permits the responding means to respond.

15. The sensing device according to claim 1, wherein said sensor(s) are comprised of weight detecting sensors that can detect the weight or pressing force of the toy means, the occupant of the baby holding means, accessory means or wrapping means or the wearer of the clothing means,
a standard degree of pressing force for each sensor is preset and stored in memory and a standard degree of pressing force for any one sensor may be the same as or different from that of the other sensor; and
the microcomputer is so programmed that, the pressing force applied to each sensor is checked through a determination as to whether the pressing force exceeds the preset standard degree or not, when the pressing force applied to any one sensor exceeds the standard degree, the microcomputer orders the responding means to produce responding signal, and when the responding means produces responding signal, it is arranged such that the response is stopped when the occupant, the wearer or the attendant (to the occupant, the wearer or the toy means), upon recognizing the responding signal, reduces the pressing force applied to the sensor.

16. The sensing device according to claim 1, wherein said baby holding means, accessory means, wrapping means, clothing means or toy means is provided with a retaining means such as a safety belt(s) and/or a shield(s), and said sensor(s) is provided to said retaining means.

17. The baby holding means according to claim 1, wherein said baby holding means comprises any one of the infant seat, child car seat, child car bed, stroller, infant bed, hammock, cradle, rocker, bassinet, shopping cart, infant carrier, baby carrying pack, swing, crib, walker, high chair, stationary infant stand, or the combination of a few of these apparatues.

18. The accessory means according to claim 1, wherein said accessory means comprises any one of the miniature of the infant seat, child car seat, child car bed, stroller, infant bed, hammock, cradle, rocker, bassinet, shopping cart, infant carrier, baby carrying pack, swing, crib, walker, high chair, stationary infant stand or the combination of a few of these apparatuses.

19. The wrapping means according to claim 1, wherein said wrapping means comprises any one of a substantially flat blanket, cushion support, pad, bunting or sheet article of flexible fabric material such as cotton, nylon, fleece, or the like, which has a front side, a back side and contiguous edges, and dimensions sufficient to fit around a child.

20. The clothing means according to claim 1 or 5, wherein said clothing means comprises any one of the diapers, covers for the diapers, shirts, unitary garments, pants, sanitary napkins, bandages and the like.

21. The toy means according to claim 1 or 6, wherein said toy means comprises any one of the dolls and toy figures provided in animal-like figures that generally resemble fanciful animals such as a dog, a cat, a rabbit, a bear or the like, human resembling figures such as warriors, heroes or the like, fantasy-type figures including cartoon and other fictional characters such as a ghost or fairy godmother or other recognizable characters.

22. The baby holding means according to claim 2, wherein said baby holding means comprises any one of the infant seat, child car seat, child car bed, stroller, infant bed, hammock, cradle, rocker, bassinet, shopping cart, infant carrier, baby carrying pack, swing, crib, walker, high chair, stationary infant stand, or the combination of a few of these apparatues.

23. The accessory means according to claim 3, wherein said accessory means comprises any one of the miniature of the infant seat, child car seat, child car bed, stroller, infant bed, hammock, cradle, rocker, bassinet, shopping cart, infant carrier, baby carrying pack, swing, crib, walker, high chair, stationary infant stand or the combination of a few of these apparatuses.

24. The wrapping means according to claim 4, wherein said wrapping means comprises any one of a substantially flat blanket, cushion support, pad, bunting or sheet article of flexible fabric material such as cotton, nylon, fleece, or the like, which has a front side, a back side and contiguous edges, and dimensions sufficient to fit around a child.

25. The clothing means according to claim 5, wherein said clothing means comprises any one of the diapers, covers for the diapers, shirts, unitary garments, pants, sanitary napkins, bandages and the like.

26. The toy means according to claim 6, wherein said toy means comprises any one of the dolls and toy figures provided in animal-like figures that generally resemble fanciful animals such as a dog, a cat, a rabbit, a bear or the like, human resembling figures such as warriors, heroes or the like, fantasy-type figures including cartoon and other fictional characters such as a ghost or fairy godmother or other recognizable characters.
